(19) United States
(12) Patent Application Publication
(10) Pub. No.: US 2016/0287426 A1
(43) Pub. Date: Oct. 6, 2016

(54) APPARATUS RESTRAINING HAND DIGIT CONTACT WITH THE FACE

(71) Applicants: Damion L. Kimmer, Bartlesville, OK (US); Joshua S. Young, Bartlesville, OK (US)

(72) Inventors: Damion L. Kimmer, Bartlesville, OK (US); Joshua S. Young, Bartlesville, OK (US)

(21) Appl. No.: 15/091,291

(22) Filed: Apr. 5, 2016

Related U.S. Application Data
(60) Provisional application No. 62/178,242, filed on Apr. 6, 2015.

(51) Int. Cl. A61F 5/37

(52) U.S. Cl. CPC A61F 5/373

(57) ABSTRACT

An apparatus for preventing hand digit contact with the face is disclosed. The apparatus includes a flexible fabric body that is configured to wrap around a user's elbow. The apparatus allows for some flexure of the elbow permitting the user the ability to perform manual functions that do not require contact with the face. A stop is positioned to span across the crook of the elbow to prevent full flexure of the elbow making it difficult for the user to have sustained contact with the face and its features.

### CROSS-REFERENCE TO RELATED APPLICATION

This application claims benefit under 35 U.S.C. §119(e) of U.S. Provisional Application having Ser. No. 62/178,242 filed Apr. 6, 2015, which is hereby incorporated by reference herein in its entirety

### FIELD

The subject disclosure relates to restraints, and more particularly, to an apparatus restraining hand digit contact with the face.

### BACKGROUND

Many people need to stop putting their hands or fingers to their face for example after dental surgery, to stop unwanted habits such as thumb sucking, or to avoid self-injury. Methods for preventing hand-to-face contact today include total arm immobilizers, protective covers for the hand or fingers, or bulky arm braces that do not fit well. The current state of the art devices and approaches are uncomfortable, do not fit well, are unhygienic, obstruct the wearer's ability to use their hands for other purposes, are ineffective, and are aesthetically unappealing. There is a need for improvement.

Known products exist generally in one of two forms: restraining devices that keep the arm straight or digit guards that cover fingers. Restraining devices on the market today are crude and cause the arm to remain nearly straight, and are therefore very uncomfortable and inhumane. Digit guards cover one or multiple digits with plastic, rubber or fabric yet allow the wearer's fingers to easily reach the mouth and simply interfere with the thumb or finger sucking. Both of these forms of products greatly interfere with otherwise normal activities such as eating or writing. Because the digit guards do not prevent the digits from reaching the mouth, they fill with slobber and germs and are therefore unhygienic. They are also unsightly and uncomfortable, so people resist the idea of wearing them in the first place thus rendering them ineffective at the outset.

As can be seen, there is a need for a device that restrains movement of the hand toward the face while allowing functional use of the hand for activities away from the face.

### SUMMARY

In one aspect of the disclosure, an apparatus for restraining hand digit contact with the face comprises a flexible fabric body configured to wrap around a user's elbow, a portion of a user's upper arm, and a portion of a user's forearm and a mechanical stop (“stop”) coupled to the flexible fabric body. The stop is positioned to span between the portion of the user's upper arm and the portion of the user's forearm across an inner side of the user's elbow and is disposed to allow less than full flexure of the user's elbow.

In another aspect of the disclosure, an apparatus for restraining hand digit contact with the face comprises a flexible fabric body configured to wrap around a user's elbow, a portion of a user's upper arm, and a portion of a user's forearm. A fastener system may secure the flexible fabric body around the user's elbow, the portion of the user's upper arm, and the portion of the user's forearm. A first strap including a first curved edge may be configured to wrap around the portion of the user's upper arm. A second strap including a second curved edge may be configured to wrap around the portion of the user's forearm. The first curved edge and second curved edge define an opening for receipt of the user's elbow when the flexible fabric body is wrapped around the user's elbow. A flexible stop is coupled to or housed in the flexible fabric body and positioned to span between the portion of the user's upper arm and the portion of the user's forearm across an inner side of the user's elbow to resist flexure of the user's elbow.

In yet another aspect of the disclosure, an apparatus restraining hand digit contact with the face comprises a flexible fabric body configured to wrap around a user's elbow, a portion of a user's upper arm, and a portion of a user's forearm. A first strap may be configured to wrap around the portion of the user's upper arm. A second strap may be configured to wrap around the portion of the user's forearm. A rigid stop is coupled to or housed in the flexible fabric body. The rigid stop is positioned to span between the portion of the user's upper arm and the portion of the user's forearm across an inner side of the user's elbow and disposed to resist flexure of the user's elbow. Also, at least one non-elastic belt may be connected to the flexible fabric body and configured to wrap around the portion of the user's upper arm or the portion of the user's forearm and over the rigid stop holding the stop in place when the flexible fabric body is wrapped around the user's elbow.

It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF THE DRAWINGS

FIG. 1 is a perspective view of an apparatus for restraining contact with the face by the user's hand in use in accordance with an aspect of the subject technology.

FIG. 2 is a top perspective view of the apparatus of FIG. 1 unwrapped.

FIG. 3 is a partial top view of the apparatus of FIG. 2.

FIG. 4 a partial bottom view of the apparatus of FIG. 2.

FIG. 5 is a cross-sectional view taken along the line 5-5 of FIG. 4.

FIG. 6 is a bottom view of the apparatus of FIG. 1 wrapped and not in use around a user's elbow.

FIG. 7 is a right side view of the apparatus of FIG. 6.

FIG. 8 is a left side view of the apparatus of FIG. 6.

FIG. 9 is a top view of the apparatus of FIG. 6.

FIG. 10 is an inside out, side view of the apparatus of FIG. 6 showing a compartment holding a stop in accordance with an embodiment of the subject technology.

FIG. 11 is a cross-sectional view taken along the line 11-11 of FIG. 10.

FIG. 12 is a side edge view of a stop in accordance with an alternate embodiment of the subject technology.

FIG. 13 is a top view of the stop of FIG. 12.

FIG. 14 is a perspective top view of the stop of FIG. 12.

### DETAILED DESCRIPTION

The detailed description set forth below is intended as a description of various configurations of the subject technology and is not intended to represent the only configurations in which the subject technology may be practiced. The appended drawings are incorporated herein and constitute a part of the detailed description. The detailed description includes specific details for the purpose of providing a thorough understanding of the subject technology. However, it will be apparent to those skilled in the art that the subject technology may be practiced without these specific details. Like or similar components are labeled with identical element numbers for ease of understanding.

In general, exemplary embodiments of the subject technology provide an apparatus that prevents or makes it very difficult to sustain contact with one's face by one's hand digits. It will be understood that “hand digits” refers to either fingers or a thumb. Yet, elements of the apparatus simultaneously permit mobility and functional use of the arm and hand wearing the apparatus. A person may still use the hand for activity that does not include touching the face. As will be appreciated, embodiments provide solutions to a multitude of applications, including the breaking of habits such as thumb sucking, finger sucking, nail biting; preventing post-surgery patients from picking at facial wounds or sutures; acting as a restraining device; etc. Although embodiments are shown and described in the context of use by children, features are equally configurable for use by adults. For example, the apparatus may impede rubbing one's eyes while typing for prolonged periods at a keyboard. The user may type normally and yet is restrained from contacting the face (eyes). Some features contemplate the sensitivities of children and yet adults would also appreciate the comfort provided by aspects disclosed herein.

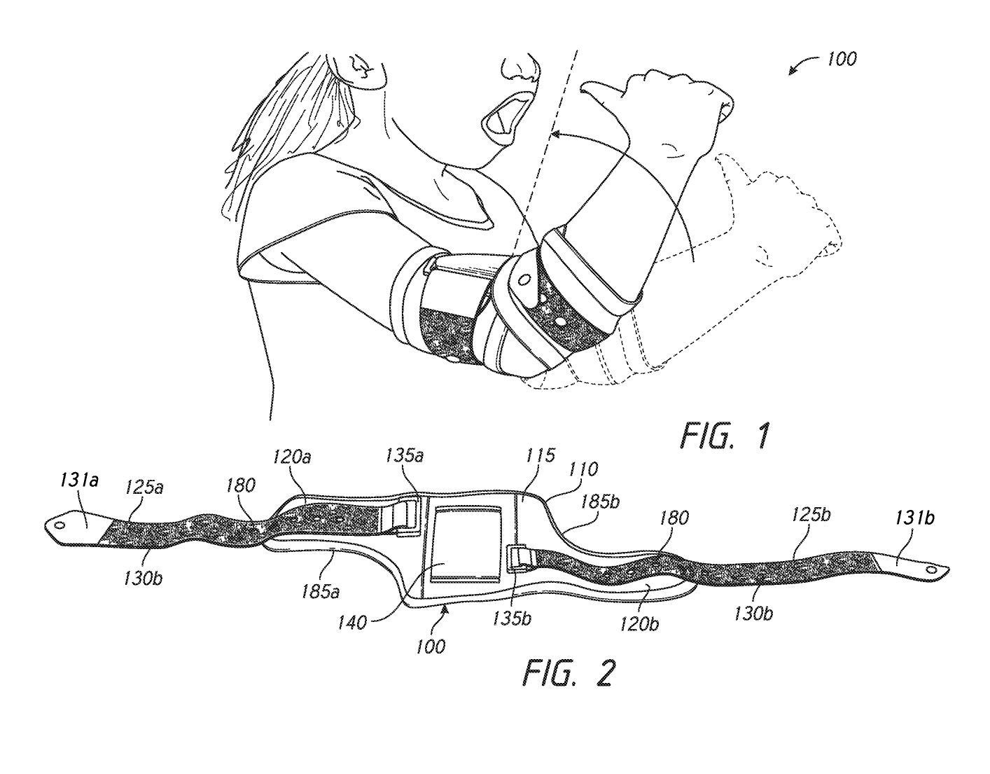

Referring now to FIG. 1, an apparatus 100 for restraining hand digit contact with the face is shown according to an exemplary embodiment. The apparatus 100 is shown in use around a child's arm. Generally, the apparatus 100 extends from a lower portion of the upper arm to an upper portion of the forearm. However, it will be understood that the extremities of the apparatus 100 may reach farther up or down the user's arm depending on the length of the arm. The apparatus 100 is configured to prevent less than full flexure at the elbow to restrain the hand on the arm wearing the apparatus 100 from reaching or at least having sustained contact with the face or facial features. For example, as shown, the apparatus 100 may be useful in restraining a child from flexing the elbow so that the child's thumb cannot be brought to the mouth or held there comfortably to suck on.

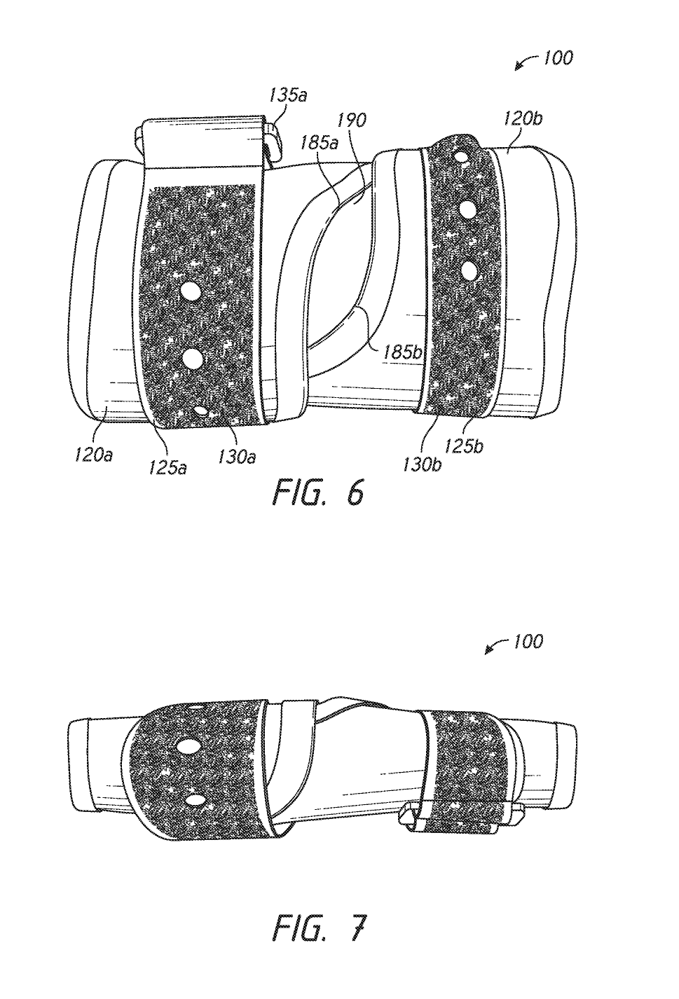

Referring now to FIGS. 1-11 in general, elements of the apparatus 100 are shown according to an exemplary embodiment. The apparatus 100 may include a flexible fabric body 110 and a stop 150 positioned in the flexible fabric body 110 to restrain movement of the forearm to the upper arm. Some embodiments may include a fastener system 125 to secure the flexible fabric body 110 to the arm. As used herein, it will be understood that a “stop” is a mechanical stop.

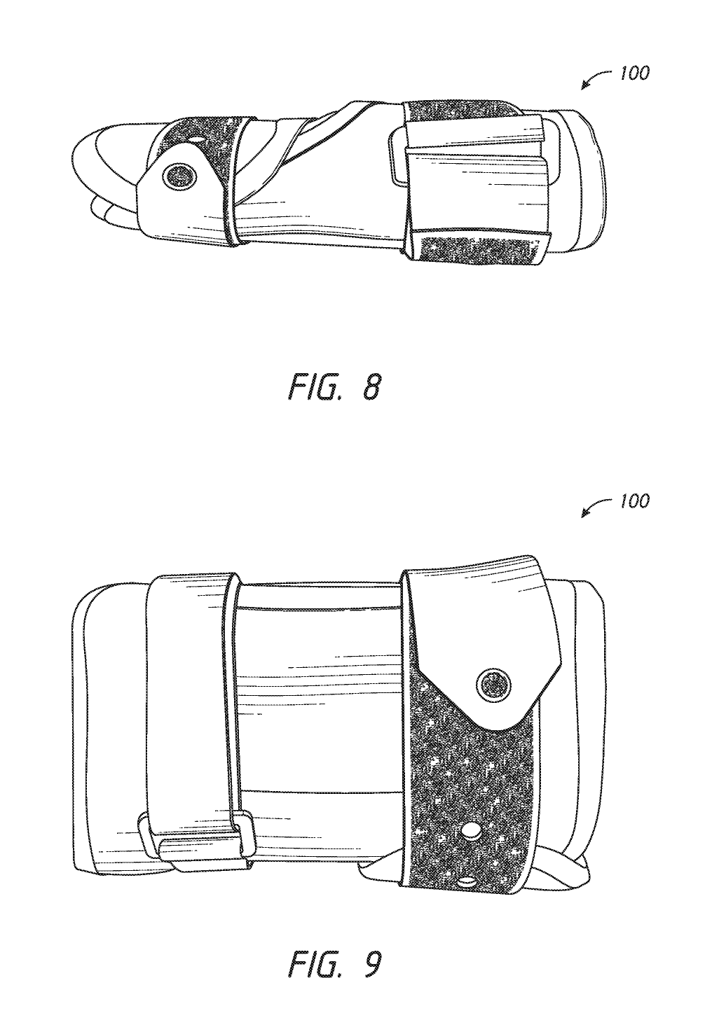

The flexible fabric body 110 may be soft, elastic material configured to wrap around the user's elbow, a portion of the user's upper arm, and a portion of the user's forearm. The flexible fabric body 110 may be for example neoprene. A portion of the flexible fabric body 110 defines an opening 190 (FIG. 6) that may receive the user's elbow. In an exemplary embodiment, the flexible fabric body 110 includes straps 120a and 120b projecting in opposite lateral directions from an upper half and lower half of a central portion 115 of the body 110. The straps 120a and 120b may include respectively curved edges 185a and 185b, which may be complementary so that when the straps 120a and 120b are wrapped around the user's elbow and adjacent arm sections, the curved edges 185a and 185b define the opening 190. Some embodiments may also include belts 125a and 125b and buckles 135a and 135b attached respectively to straps 120a and 120b to secure the stop 150 in place in the elbow area as the apparatus 100 is worn. Some embodiments may include a hook and loop system that comprises a loop area (130a; 130b) on respective belts 125a; 125b that secure to a hook area (131a; 131b) on ends of the respective belts, after being looped through buckles (135a; 135b) on respective belts, as shown in FIG. 8-9.). Some embodiments may include a loop system on the central portion 115. The hook and loop systems on the straps 120a and 120b secure the straps 120a and 120b to the fabric body 110 and in turn, secure the apparatus 100 around the user's arm. Since user arm circumference varies, the loop portion extends as needed around the flexible fabric body 110 and over the top of the stop 150, so that the apparatus 100 is adjustable to different arm sizes when securing the stop 150 into place by holding the edges of the stop 150 in position against the arms (upper arm and forearm). FIGS. 6-9 show the apparatus 100 as it would be wrapped around the elbow in use. Some embodiments may include holes 180 in the belts 125a and 125b which when the belts 125a and 125b are laced through the buckles 135a and 135b and tightened into place, can be secured into position by a locking mechanism such as a cable tie, a snap, etc. being fed through the desired hole 180.

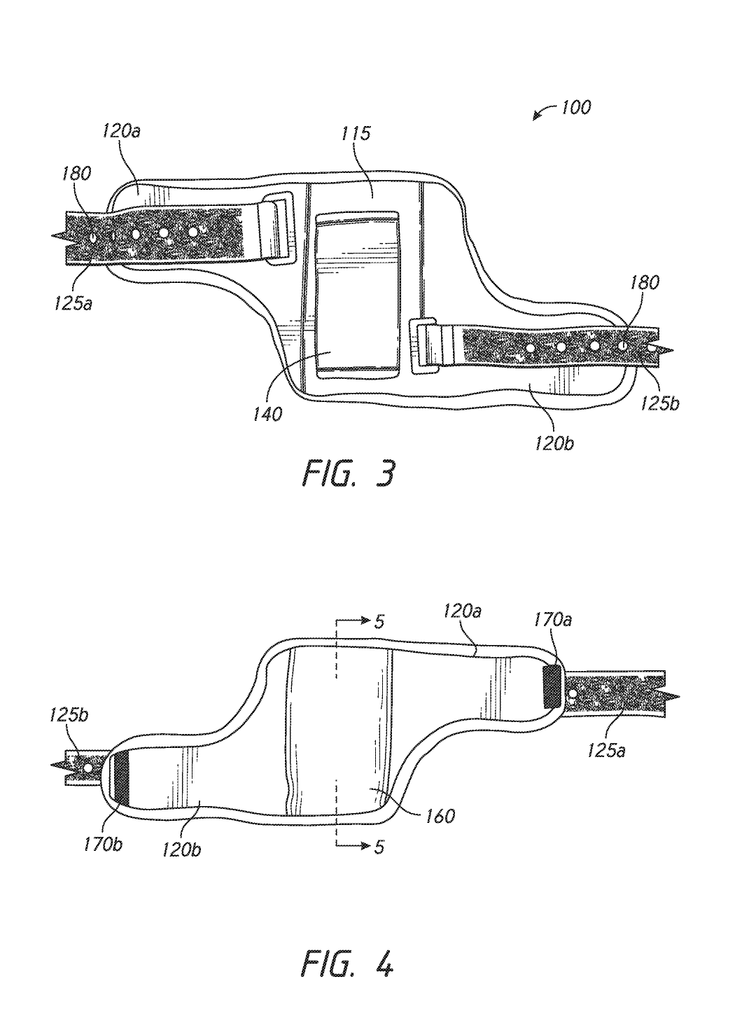

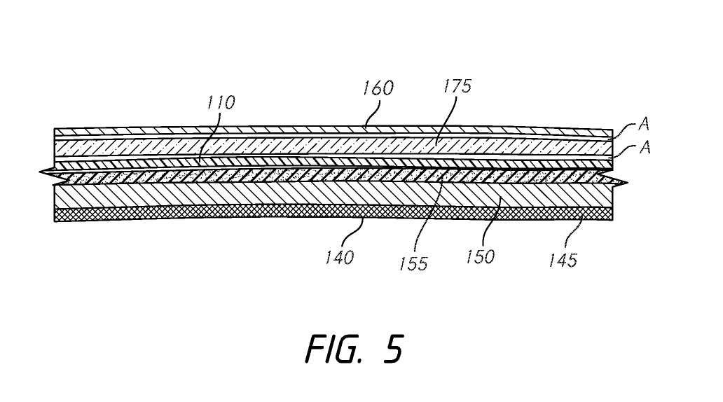

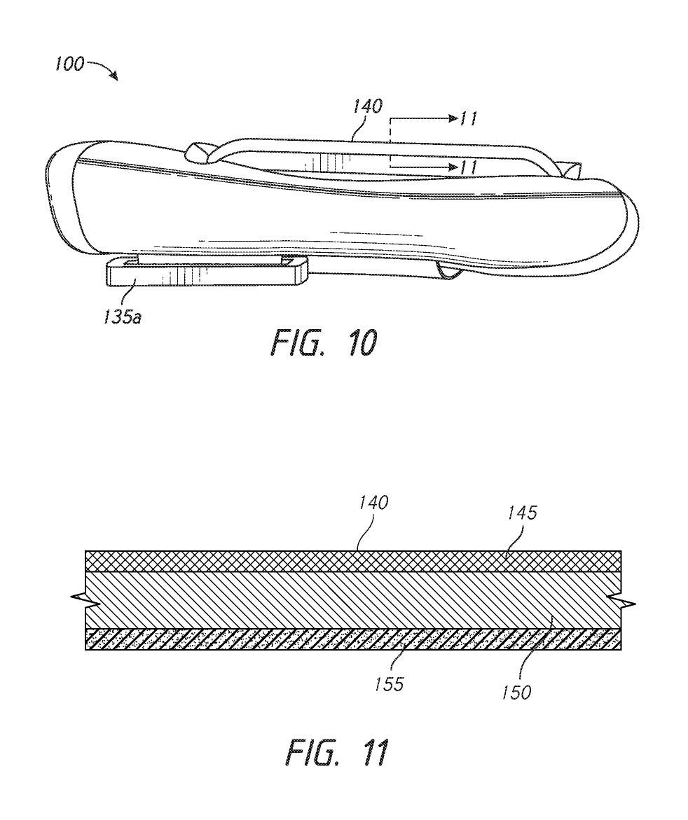

Referring now to FIGS. 3 and 10-11, the apparatus 100 incorporates a stop 150 positioned on an outer or top surface of the central portion 115. The stop 150 may be for example a rigid or semi-rigid flat plastic or metal plate. In general, ends of the stop 150 are positioned to contact the upper arm and the forearm when the apparatus 100 is worn. The stop 150 may be positioned lengthwise to be perpendicular to the direction of projection of the straps 120a and 120b. Some embodiments house the stop 150 inside of a compartment 140 sewn by the compartment's edges to the central portion 115 so that at least a middle section of the compartment is detached from the central portion 115 (FIG. 10). Some embodiments may include a loop system 145 on an exterior facing surface of the compartment 140 and a fabric layer 155 on an interior facing surface of the compartment 140 (see FIGS. 5 and 11). The loop system 145 may lock in the hook systems on the straps 170a and 170b to allow securing the flexible fabric body 110 around (similar to embodiments using a loop system on the central portion 115 described above). When the apparatus 100 is worn, the stop 150 is positioned between the portion of the user's upper arm and the portion of the user's forearm across an inner side of the user's elbow (the crook) to wedge the two arm portions from each other allowing less than full flexure of the user's elbow. For embodiments using a centrally detached compartment 140, some flexure at the elbow causes the compartment 140 and internal stop 150 to form a triangular gap with the intersection of the forearm and upper arm. Embodiments using a flexible plastic stop 150 may yield more between the contact points of the forearm and upper arm than for example, rigid metallic plate stops 150.

Referring now to FIGS. 4 and 5, the apparatus 100 may include a pad 160 on an interior or bottom side of the central portion 115 to provide the inner side of the user's elbow protection from abrasion. The pad 160 may house a cushion 175 to provide comfort to the user wearing the apparatus 100. The cushion 175 may be longer than the stop 150 so that ends of the cushion 175 may dampen pressure between contact of the stop 150 with the user's upper arm and/or forearm. The pad 160 may include soft fabric to mitigate against abrasion which may be especially appreciated by the sensitive skin of children.

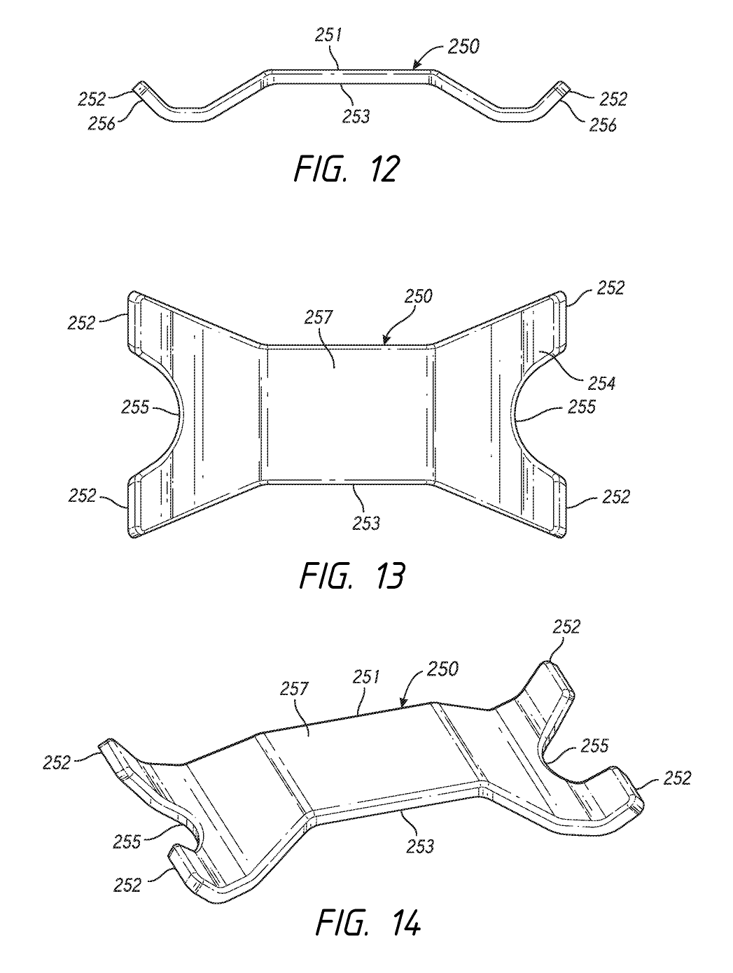

Referring now to FIGS. 12-14, an alternate embodiment of a stop 250 is shown. It will be understood that the stop 250 may be used instead of the stop 150 with respect to the remaining elements of the apparatus 100 described above. The stop 250 may be a semi-rigid flexible material such as hard plastic or resilient metals. Exemplary embodiments of the stop 250 may flex without allowing full flexure of the user's arm and may provide a counter force (for example a spring bias) to resist flexing and return the stop 250 to a default unflexed state. The stop 250 may include a central bridge 251 with an arched underside 253. An exemplary embodiment includes bowed distal ends 256 (one or both) to contact the user's upper arm and forearm. The distal ends 252 may be curved upward so that bottom surfaces of the ends 256 make contact with or resist against the upper arm and forearm (when the apparatus 100 is worn). Some embodiments may include a curved recess 255 in the center of the distal ends 256 and prongs 252 on the sides of the curved recess 255. In operation, as the user attempts to flex his or her arm, the upper surface 257 of the bridge 251 will bow (not shown). The distal ends 256 flex inward so that top surfaces 254 of distal ends 256 bend slightly to face each other resisting pressure placed upon the bottom surfaces of ends 256 by the user's upper arm and forearm. As will be appreciated the curved recesses 255 may receive a portion of the upper arm and forearm and may work in conjunction with the prongs 252 to hold onto respective arm portions and resist rotational or lateral displacement of the stop 250 when the user attempts to flex the arm. The prongs 252 may provide additional counter spring action by semi-flexing in response to contact with the upper arm and/or forearm and resisting flexure of the stop 250.

It may be appreciated that aspects of the apparatus 100 may be worn discretely around an arm and does not interfere with normal activities, yet the apparatus prevents the digits from reaching the face altogether. This is a significant contrast when compared to prior art devices that either keep the arm straight or simply put a barrier around the digits as they are inserted into the mouth. Further, the apparatus 100 can be discretely worn under the clothes on either the right or left arm, is comfortable and adjustable to different body sizes, and is easy to use. In addition, hygiene is preserved because the apparatus 100 is positioned when worn to avoid the mouth and hence does not collect saliva. All of these benefits increase the frequency of its application and thus its effectiveness.

Those of skill in the art would appreciate that various components may be arranged differently (e.g., arranged in a different order, or partitioned in a different way) all without departing from the scope of the subject technology.

The previous description is provided to enable any person skilled in the art to practice the various aspects described herein. The previous description provides various examples of the subject technology, and the subject technology is not limited to these examples. Various modifications to these aspects will be readily apparent to those skilled in the art, and the generic principles defined herein may be applied to other aspects. For example, some embodiments may integrate the stop 150 (or 250) within the interior of the central portion 115 instead of the compartment 140 and yet the stop 150 (or 250) may still restrain flexure between the forearm and upper arm. In addition, while embodiments are described above as a flexible fabric body 110 being flat and wrapped into securement around the arm, it will be understood that other embodiments may include a sleeve that can be slid on over the elbow area incorporating a stop that spans across the elbow from the upper arm to the forearm as described above. Thus, the claims are not intended to be limited to the aspects shown herein, but is to be accorded the full scope consistent with the language claims, wherein reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

Terms such as “top,” “bottom,” “front,” “rear,” “above,” “below” and the like as used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference. Similarly, an item disposed above another item may be located above or below the other item along a vertical, horizontal or diagonal direction; and an item disposed below another item may be located below or above the other item along a vertical, horizontal or diagonal direction.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

The word “exemplary” is used herein to mean “serving as an example or illustration.” Any aspect or design described herein as “exemplary” is not necessarily to be construed as preferred or advantageous over other aspects or designs.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. §112, sixth paragraph, unless the element is expressly recited using the phrase “means for” or, in the case of a method claim, the element is recited using the phrase “step for.” Furthermore, to the extent that the term “include,” “have,” or the like is used in the description or the claims, such term is intended to be inclusive in a manner similar to the term “comprise” as “comprise” is interpreted when employed as a transitional word in a claim.

What is claimed is:

1. An apparatus for restraining hand digit contact with the face, comprising:
a flexible fabric body configured to wrap around a user's elbow, a portion of a user's upper arm, and a portion of a user's forearm; and
a mechanical stop coupled to the flexible fabric body, the stop positioned to span between the portion of the user's upper arm and the portion of the user's forearm across an inner side of the user's elbow and disposed to allow less than full flexure of the user's elbow when the flexible fabric body is wrapped around the user's elbow.

2. The apparatus of claim 1, wherein the stop is a plate including a first end disposed for contact with the portion of the user's upper arm and a second end disposed for contact with the portion of the user's forearm.

3. The apparatus of claim 1, further comprising a first strap configured to wrap around the portion of the user's upper arm and a second strap configured to wrap around the portion of the user's forearm.

4. The apparatus of claim 3, further comprising a fastener system, the fastener system including a hook and loop system for each of the first and second strap, a hook portion of the hook and loop system positioned on an interior side of the flexible fabric body for each strap and a loop portion positioned on a surface side of each strap.

5. The apparatus of claim 3, further comprising a first curved edge on the first strap and a second curved edge on the second strap, the first and second curved edges configured to define an opening for receipt of the elbow when the flexible fabric body is wrapped around the user's elbow.

6. The apparatus of claim 1, further comprising a pad on an interior side of the flexible fabric body disposed to provide the inner side of the user's elbow protection from abrasion.

7. The apparatus of claim 6, further comprising a cushion being longer than the stop and disposed in the flexible fabric body to dampen pressure between the stop and the user's portion of the upper arm and/or the user's portion of the forearm.

8. The apparatus of claim 1, further comprising at least one non-elastic belt and configured to wrap over the stop distal ends thereby holding the stop in place against the user's upper arm or forearm when the flexible fabric body is wrapped around the user's elbow.

9. The apparatus of claim 1, further comprising a compartment coupled to the flexible fabric body, the compartment housing the stop, the compartment positioned on an inner side of the user's arm when the flexible fabric body is wrapped around the user's elbow and positioned to span between the portion of the user's upper arm and the portion of the user's forearm across an inner side of the user's elbow.

10. The apparatus of claim 1, wherein the stop is replaceable with different sized stops to control an amount of flexure in the user's elbow.

11. The apparatus of claim 1, wherein the stop is semi-rigid and configured to flex when the user's elbow is flexed and includes a spring bias to provide a counter force to the flex to restore the stop to a default unflexed status.

12. An apparatus restraining hand digit contact with the face, comprising:
a flexible fabric body configured to wrap around a user's elbow, a portion of a user's upper arm, and a portion of a user's forearm;
a fastener system configured to secure the flexible fabric body around the user's elbow, the portion of the user's upper arm, and the portion of the user's forearm;
a first strap including a first curved edge configured to wrap around the portion of the user's upper arm;
a second strap including a second curved edge configured to wrap around the portion of the user's forearm, wherein the first curved edge and second curved edge define an opening for receipt of the user's elbow when the flexible fabric body is wrapped around the user's elbow; and
a flexible mechanical stop coupled to or housed in the flexible fabric body, the flexible stop positioned to span between the portion of the user's upper arm and the portion of the user's forearm across an inner side of the user's elbow and disposed to resist flexure of the user's elbow.

13. The apparatus of claim 12, wherein the first strap projects laterally in a first direction from the flexible fabric body and the second strap projects laterally in a second direction from the flexible fabric body.

14. The apparatus of claim 12, wherein the stop is disposed as a wedge between the portion of the user's upper arm and the portion of the user's forearm in response to the user flexing at the elbow.

15. The apparatus of claim 12, wherein the stop includes a recess on at least one end of the stop, the recess configured to receive a section of either the portion of the user's upper arm or the portion of the user's forearm.

16. The apparatus of claim 12, wherein the stop includes a central arch configured to flex when the user's elbow is flexed, and configured to include a spring bias to provide a counter force to the flex to restore the stop to a default unflexed status.

17. The apparatus of claim 12, wherein the stop includes a curved distal end configured to contact either the portion of the user's upper arm or the portion of the user's forearm.

18. An apparatus restraining hand digit contact with the face, comprising:
a flexible fabric body configured to wrap around a user's elbow, a portion of a user's upper arm, and a portion of a user's forearm;
a first strap configured to wrap around the portion of the user's upper arm;
a second strap configured to wrap around the portion of the user's forearm;
a semi-rigid mechanical stop coupled to or housed in the flexible fabric body, the semi-rigid stop positioned to span between the portion of the user's upper arm and the portion of the user's forearm across an inner side of the user's elbow and disposed to resist flexure of the user's elbow; and
at least one non-elastic belt connected to the flexible fabric body and configured to wrap around the portion of the user's upper arm or the portion of the user's forearm and over the semi-rigid stop holding the stop in place when the flexible fabric body is wrapped around the user's elbow. The apparatus of claim 18, further comprising a hook and loop system on the non-elastic belt to secure the belt over the semi-rigid stop.

20. The apparatus of claim 18, wherein the semi-rigid stop is a plate wedged between the portion of the user's upper arm and the portion of the user's forearm when the flexible fabric body is wrapped around the user's elbow.
